(19) United States
(12) Patent Application Publication
(10) Pub. No.: US 2024/0008876 A1
(43) Pub. Date: Jan. 11, 2024

(54) ELECTROSTATIC DELIVERY OF SURGICAL MATERIAL

(71) Applicant: GYRUS ACMI, INC. D/B/A OLYMPUS SURGICAL TECHNOLOGIES AMERICA, Westborough, MA (US)

(72) Inventors: Kester Julian Batchelor, Mound, MN (US); Charles A. Baker, Rogers, MN (US); Jordan N. Milford, Bethlehem, PA (US); Joey Magno, Dudley, MA (US)

(21) Appl. No.: 18/342,223

(22) Filed: Jun. 27, 2023

Related U.S. Application Data
(60) Provisional application No. 63/367,965, filed on Jul. 8, 2022.

(51) Int. Cl. A61B 17/12; A61B 1/313

(52) U.S. Cl. CPC A61B 17/12181; A61B 1/3132; A61B 17/3415

(57) ABSTRACT

A method for delivering surgical material comprises inserting a delivery device into an anatomic area, coupling a surgical material delivery system to the delivery device, the surgical material delivery system having a reservoir of surgical material, positioning an anatomic electrode relative to the anatomic area, applying a first charge to the anatomic electrode, dispensing surgical material from the delivery device, applying a second charge to surgical material leaving the delivery device opposite the first charge, and delivering charged surgical material to the anatomic area proximate the anatomic electrode via a directional-oriented electrostatic field. A surgical system comprises an insertion shaft comprising a passageway and a discharge opening, a first electrode connected to the insertion shaft configured to impart electrical charge to surgical material flowing through the passageway, and a second electrode connected to the insertion shaft configured to impart electrical charge to tissue in contact with the second electrode.

### PRIORITY CLAIM

This application claims the benefit of priority to U.S. Provisional Patent Application Ser. No. 63/367,965, filed Jul. 8, 2022, the contents of which are incorporated herein by reference.

### TECHNICAL FIELD

This document pertains generally, but not by way of limitation, to surgical systems and methods for preparing an anatomic site for surgery. More specifically, but not by way of limitation, the present application relates to systems and methods for delivering surgical material, such as hemostat material, to a surgical site to inhibit or stop bleeding.

### BACKGROUND

Many surgical procedures involve the treatment or removal of target tissue, e.g., diseased, potentially diseased or otherwise unwanted tissue, located inside of a patient. As such, some of these procedures require access to the internal anatomy of the patient via an open procedure or through a smaller opening in minimally invasive (e.g., laparoscopic) procedures. In some endoscopy cases, the patient anatomy is accessed through the mouth or anus, as well as any natural orifice as can be used in urology, gynecology, ear-nose-throat (ENT) procedures, without producing an opening or incision in the patient to reach an internal cavity or duct within the patient, such as the gastrointestinal (GI) tract. These endoscopy procedures can be referred to as endolumenal procedures because the procedures take place inside a tube, duct or hollow organ in the body. Some endolumenal procedures involve the removal of tissue from a tissue wall forming the duct or cavity. As such, it can be desirable in these and other applications to administer a hemostat material, such as a powdered or liquid clotting agent, to limit or stop bleeding to facilitate performance of the procedure and healing of the patient.

### OVERVIEW

The present inventors have recognized, among other things, that problems to be solved with hemostat delivery devices include the difficulty in providing simple to use systems that provide a user-friendly experience. For example, some hemostat materials comprise liquids that are delivered with difficult-to-use, manually operated syringes. Some hemostat powder delivery systems operate with a pump that is located at the hospital or facility at which the procedure is performed. However, such pumps require a large initial expenditure by the procedure provider. Some hemostat delivery systems operate using pressurized air or CO2 provided by the facility. However, the pressures at which these gases operate can fluctuate based on building conditions, such as how much of the gas other functions of the facility are using at the time of the procedure. Additionally, other handheld hemostat powder delivery devices utilize compressed gas cartridges that provide pressurized gas over a wide range of pressures. For example, the cartridge can provide an initially high pressure that gradually tapers off as propellant in the cartridge diminishes. The initially high pressure can often be too high, resulting in excessive spray of the hemostat powder onto areas where it is not intended to reach, such as anatomy away from the bleeding or a scope being used in the procedure, thereby potentially obstructing lenses and lumens of the scope. Additionally, the present inventors have recognized that even with the use of a pressure limiting valve, the performance of the compressed gas canister still diminishes over time and provides an inconsistent user experience.

In summary, two major issues persist with the use of pressurized gas cartridges for delivery of clotting agents, such as hemostat powder: 1) The initial pressure can be too high causing powder to fill the lumen of the anatomy and loss of visibility due to powder being dispersed in the air and obstructing lenses (which physician refers to as a “white-out”); and 2) the powder can attach itself to places that it is not intended to attach such as the endoscope or areas of the bowel that do not need to be treated. The present inventors have recognized that, as the pressure in the propellant cartridge reduces, the physician can have better control and can direct the hemostat to the appropriate area, but the continuously decreasing pressure can affect a consistent user experience. Thus, the present inventors have recognized that it is desirable for a hemostat delivery device to provide a consistent pressure over a period of time to allow for delivery at an appropriate level in a predictable manner. Furthermore, the present inventors have recognized that it is desirable to be able to control the trajectory of hemostat material to better apply the material to a desired treatment area without undesirably covering other areas.

The present subject matter can provide solutions to these problems and other problems, such as by providing surgical substance or surgical material delivery devices and systems, such as hemostat material delivery devices and systems, that provide a cost-effective, user-friendly experience. In examples, the surgical substance can comprise hemostat material, such as hemostat powder or hemostat liquid or fluid. In additional examples, the surgical substance can comprise collagen, medicants, therapeutic substances, dyes or coloring agents, adhesives, and other substances that can be used for therapeutics, diagnostics and other applications. In particular, the present subject matter can provide a hemostat delivery system that can deliver hemostat material, such as a powder, via an electrostatic guide system that can guide hemostat material to a target anatomic site, which thereby eliminates or reduces the “white-out” effect and reduces instances of the powder attaching to unintended or undesirable locations including other anatomic areas and the hemostat delivery system. The delivery system can apply a charge to hemostat material emitted from a delivery instrument, such as a catheter, to thereby draw the charged hemostat material to an oppositely charged or neutral target anatomy site. The hemostat material can thus be delivered at pressures below where white out conditions occur and at a level, e.g., volume, so that the hemostat material can be delivered in a consistent manner over a prolonged period of time where the user intends the material to be delivered. Furthermore, the electrically-charged hemostat material can be directionally oriented or aimed such that only a desired target area receives hemostat material rather than an entire anatomic area. Directionally orienting surgical material can reduce the amount of surgical material that is undesirably used and can reduce the time it takes to apply surgical material to the desired location, thereby saving costs associated with performing a procedure.

In an example, a surgical material delivery system can comprise an elongate insertion shaft comprising a passageway extending at least partially through the elongate insertion shaft and a discharge opening fluidly connected to the passageway, a first electrode connected to the elongate insertion shaft configured to impart an electrical charge to surgical material flowing through the passageway, and a second electrode connected to the elongate insertion shaft configured to impart an electrical charge to tissue in contact with the second electrode.

In another example, a method for delivering a surgical material to an internal lumen of a patient can comprise inserting a delivery device into an anatomic area, coupling a surgical material delivery system to the delivery device, the surgical material delivery system having a reservoir of a surgical material, positioning an anatomic electrode relative to the anatomic area, applying a first charge to the anatomic electrode, dispensing surgical material from the delivery device, applying a second charge to surgical material leaving the delivery device, the second charge opposite the first charge, and delivering charged surgical material to the anatomic area proximate the anatomic electrode via a directional-oriented electrostatic field.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is schematic view of an operating room equipped with a hemostat material delivery system having an electrostatic guide system.

FIG. 2 is a close-up schematic view of positively charged hemostat powder flowing from a delivery catheter toward negatively charged target tissue.

FIG. 3 is a schematic illustration of an imaging and control system comprising a control unit suitable for use with the hemostat material delivery system and electrostatic guide system of FIGS. 1 and 2.

FIG. 4 is schematic diagram of the control unit of FIG. 3 connected to an endoscope capable of receiving a hemostat material delivery catheter and capable of having native hemostat material delivery capabilities.

FIG. 5A is an end view of the endoscope of FIGS. 3 and 4 illustrating various components of a functional section including a camera module.

FIG. 5B is a cross-sectional view taken along section 5B-5B of FIG. 5A showing components of the camera module.

FIG. 6A is a schematic view of an endoscopy system comprising a hemostat material delivery system including an electrostatic guide system comprising a reservoir for hemostat material and various electrodes.

FIG. 6B is a perspective view of the endoscope of FIG. 6A showing a medical instrument extending therefrom.

FIG. 6C is a perspective view of a hemostat instrument suitable for use with the electrostatic hemostat material delivery system of FIG. 6A.

FIG. 7 is a perspective view of a hemostat instrument suitable for use with the electrostatic hemostat material delivery system of the present disclosure comprising a retractable anatomy electrode.

FIG. 8A is a schematic exploded view of a distal electrode device configured for placement at a distal end of a scope.

FIG. 8B is a schematic assembled view of the distal electrode device of FIG. 8A attached to the scope.

FIG. 9 is a schematic view of the distal electrode device attached to a distal end of a scope to dispense hemostat material to target tissue.

FIG. 10 is schematic illustration of a trocar device system configured to generate an electrical charge for use with an electrostatic hemostat material delivery system.

FIG. 11 is a perspective view of an electrode mat shaped to mate with particular anatomic areas.

FIG. 12 is a cross-sectional view of an electrode mat shaped to conform to anatomic contours.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

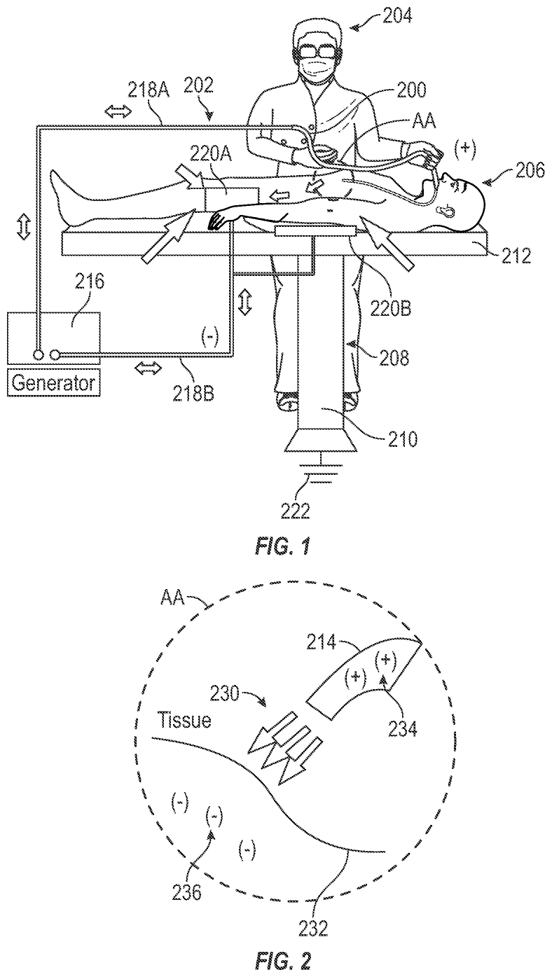

FIG. 1 is a is schematic view of an operating room equipped with hemostat material delivery system 200 having electrostatic guide system 202 being used by surgeon 204 on patient 206. Patient 206 can be located on table 208 of the operating room. Table 208 can comprise stand 210 and bed 212. Hemostat material delivery system 200 can comprise catheter 214 that can be delivered to internal anatomic locations in patient 206. Electrostatic guide system 202 can comprise generator 216, first lead 218A, second lead 218B and pad 220A. Stand 210 of table 208 can comprise ground 222 for electrostatic guide system 202.

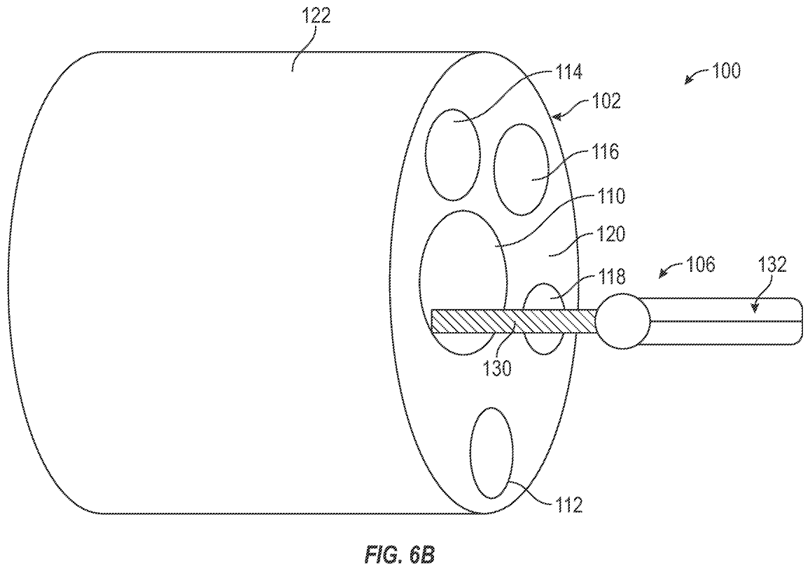

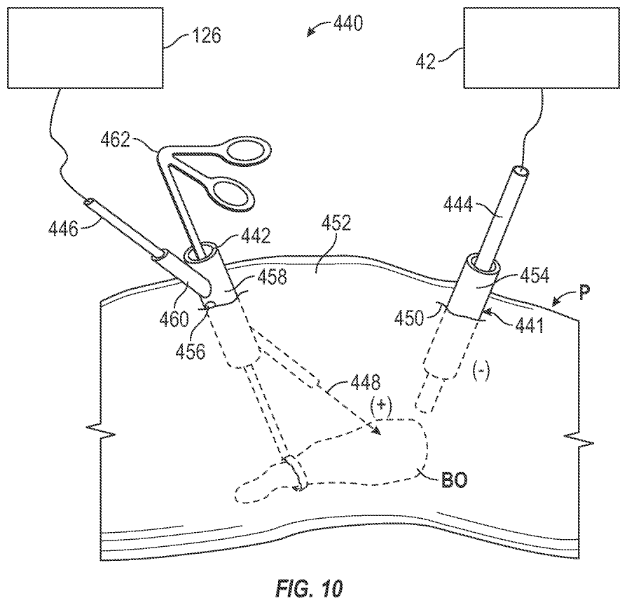

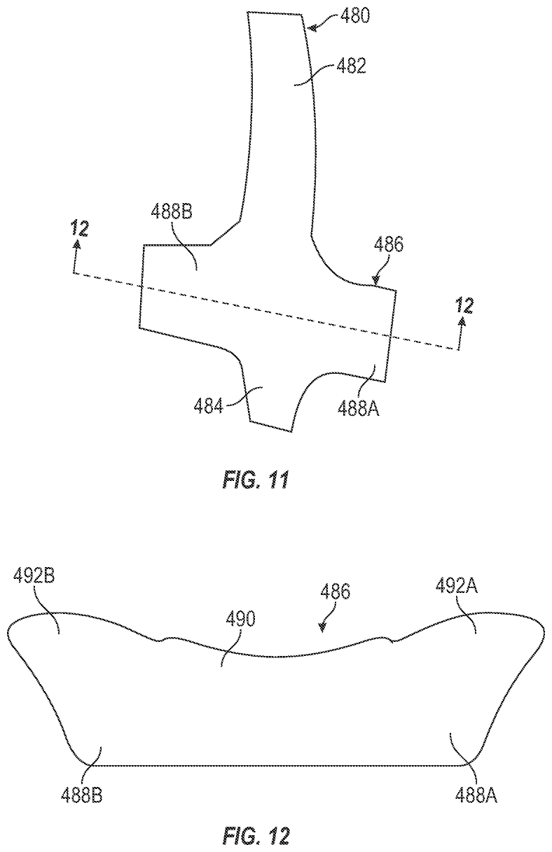

Hemostat material delivery system 200 can be configured to deliver a hemostat material, such as a clotting agent, to patient 206 via catheter 214. As discussed with reference to FIGS. 6A-6C, catheter 214 can be connected to a source of a powdered hemostat material, such as hemostat material reservoir 126 of FIG. 6A. Generator 216 can be configured to generate opposite electrical charges at catheter 214, first pad 220A and second pad 220B. As such, powder leaving catheter 214 can acquire a first charge and be attracted to tissue proximate pad 220A having the opposite charge. For example, generator 216 can apply a positive charge to catheter 214 using first lead 218A and a negative charge to pad 220A using second lead 218B. Ground 222 can induce a neutral charge to patient 206, which can facilitate charged hemostat material flowing toward patient 206. Pad 220A can be positioned between patient 206 and bed 212 in the general location of where catheter 214 is to be used to dispense hemostat material. Ground 222 and pad 220A can facilitate flow of charged hemostat material generally toward anatomy of patient 206, thereby preventing hemostat material particles from remaining airborne and sticking to catheter 214. For example, electrification of any portion of patient 206 can provide a polarity difference between the oppositely charged hemostat material. Thus, the oppositely charged hemostat material can disperse from catheter 214 in various direction to various portions of the anatomy of patient 206, which is acceptable to remove the hemostat material from the air. The anatomy of patient 206 will have a greater opposite charge closer to pad 220A. As such, the greater the distance between catheter 214 and pad 220A, the less electrostatic attraction will be generated. Thus, pad 220B can be used proximate a specific anatomic target tissue to draw hemostat material in a specific direction from catheter 214 rather than just generally away from catheter 214 and toward any tissue. Thus, pad 220B can be positioned between patient 206 and bed 212 in a specific location proximate an organ or anatomic feature where hemostat material is desired to be used, such as proximate a location where bleeding or hemorrhaging is occurring. Pad 220B can be a local pad that is smaller in area than pad 220A. As discussed with reference to FIGS. 11 and 12, pad 220B can be shaped to mate with particular exterior anatomic features of patient 206, such as a shoulder or lumbar region of a spinal column.

Generator 216 can generate an electric field between leads 218A and 218B. Generator 216 can comprise a generator used to perform other surgical functions, such as providing energy for ablation or cauterization. In examples, generator 216 can comprise an electrosurgical unit (ESU) monopolar generator, such as an ESG-400 commercially available from Olympus Surgical Technologies.

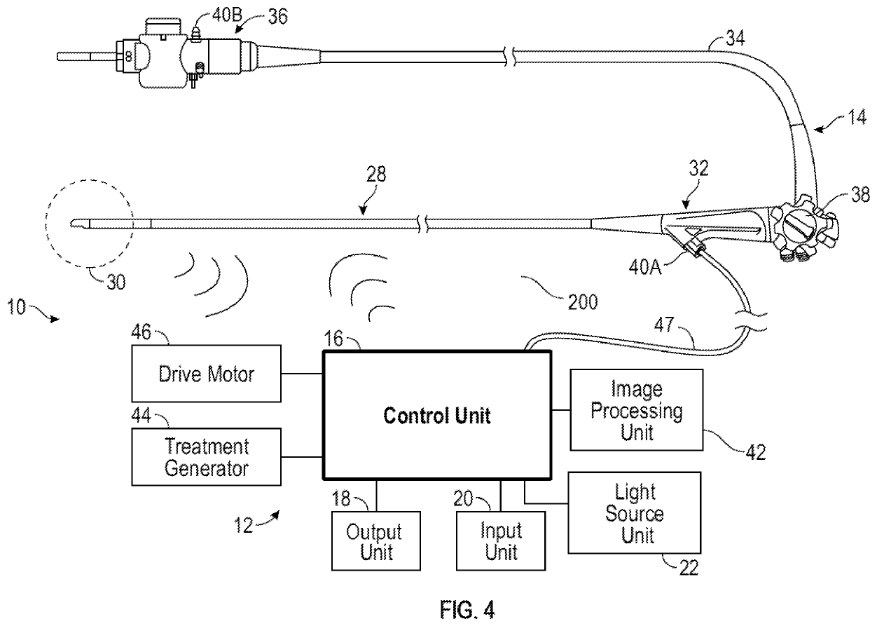

Catheter 214 can be inserted directly into patient 206 in an open procedure or inserted into patient 206 using an endoscope in a minimally invasive procedure. Catheter 214 can be connected to a device for propelling or pushing hemostat material through catheter 214, such as a pump, including motive device 124 of FIG. 6A. Application of pressure and electric charge to the hemostat material can be controlled by an operator using a user-interface device such as a button or lever on catheter 214. In examples, electrostatic charge can be automatically applied when the user-interface device of catheter 214 is actuated to deliver hemostat material. Additionally, separate user-interface devices can be provided for each of delivering and electrically charging the hemostat material. As discussed herein, in various examples, hemostat delivery capabilities and controls can be incorporated directly into a scope, such as endoscope 14 of FIG. 3 or endoscope 104 of FIG. 6A.

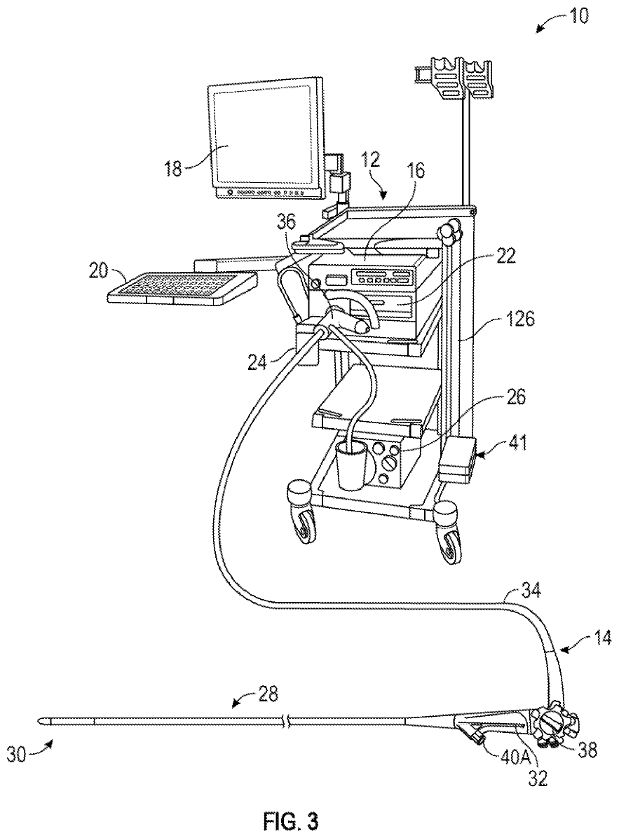

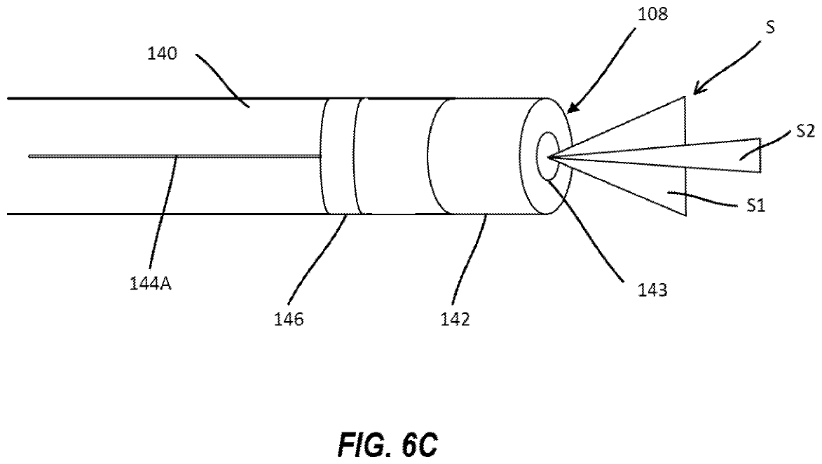

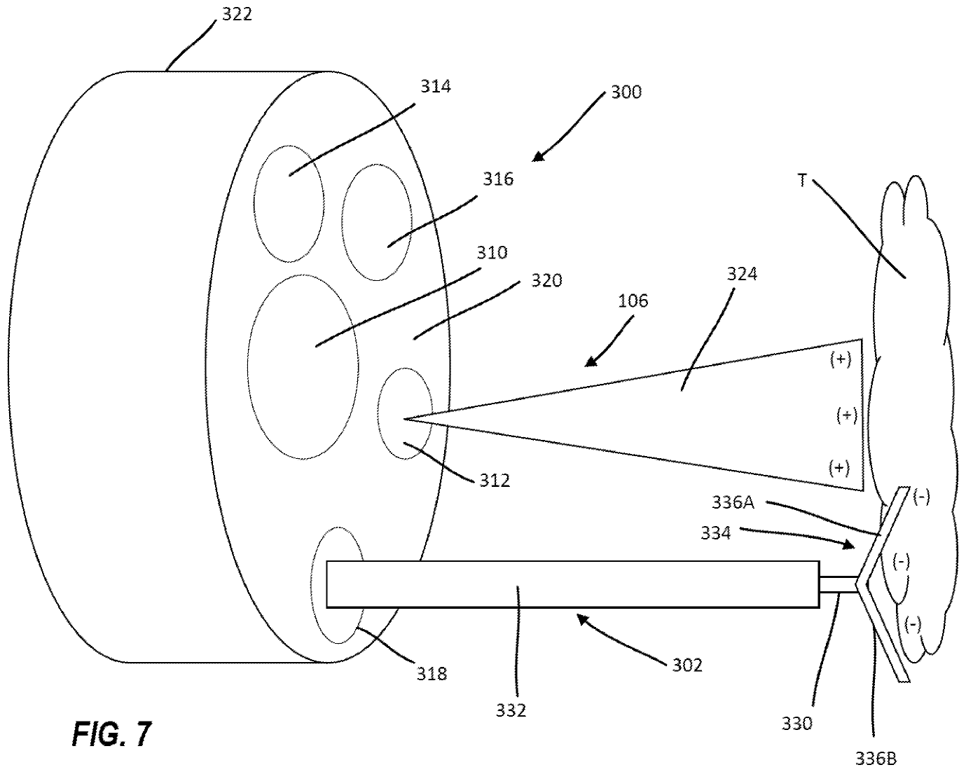

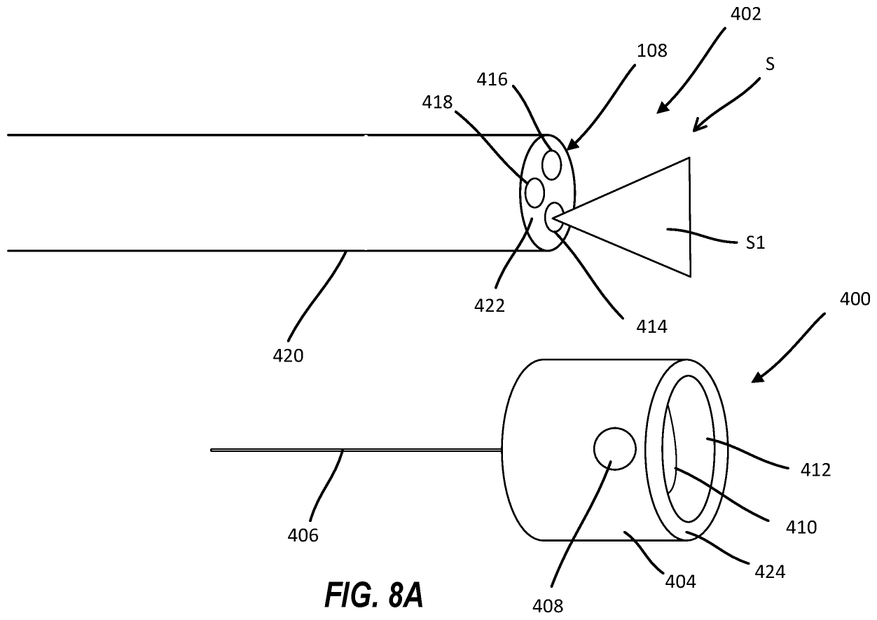

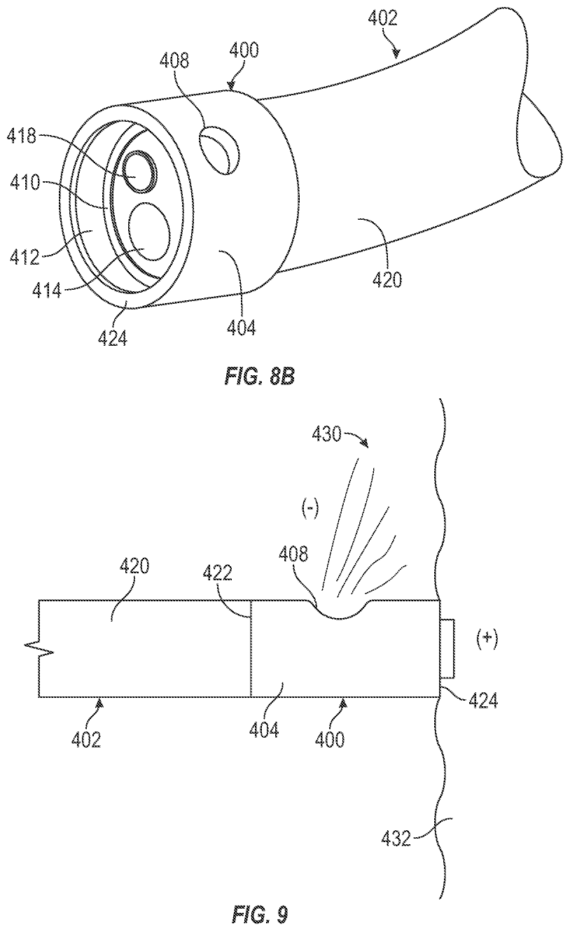

FIG. 2 is a close-up schematic view of the distal end portion of catheter 214 taken at callout AA of FIG. 1. FIG. 2 illustrates positively charged hemostat material 230 flowing from catheter 214 toward negatively charged target tissue 232. Positive charge 234 can be generated at the distal end of catheter 214 and negative charge 236 can be generated proximate target tissue 232. Positive charge 234 can be generated by one or more electrodes positioned on catheter 214 connected to lead 218A and negative charge 236 can be generated by pads 220A and 220B connected to electrode 218B, as is discussed in greater detail with reference to FIG. 6A. In another example, negative charge 236 can be generated by an electrode extending from catheter 214, as explained with reference to FIG. 7. In additional examples, negative charge 236 can be generated with a gel, such as electrode material 150 of FIG. 6A, or another conducing, biocompatible material applied to target tissue 232 internal to the patient. As shown in FIGS. 8A-9, an electrode can be positioned around a distal end face of a scope to charge tissue. As shown in FIG. 10, a trocar device can be used as an electrode to charge tissue. In yet another example, negative charge 236 can be a result of naturally occurring charge in certain anatomic features, such as blood. In the various examples, positively charged hemostat material 230 can be attracted to negatively charged target tissue 232 to facilitate hemostat material 230 locating target tissue 232. In additional examples, hemostat material 230 can be negatively charged and target tissue 232 can be positively charged or neutral.

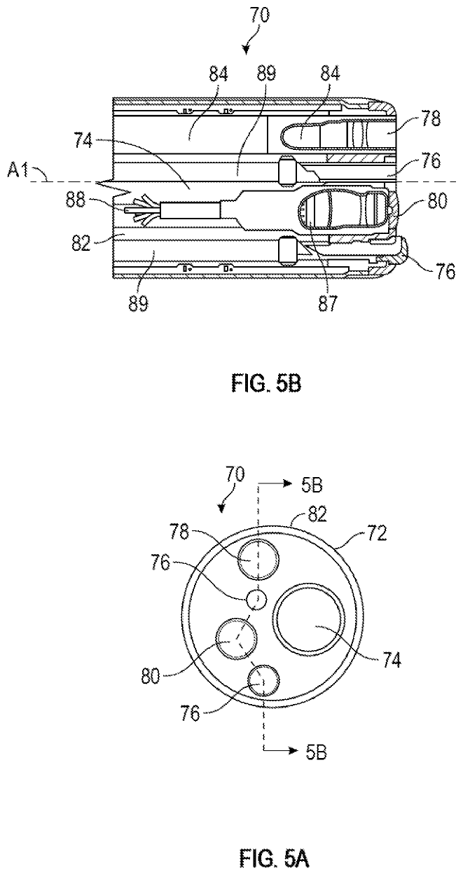

FIG. 2 is a schematic diagram illustrating a method of delivering hemostat material 230 to an internal anatomic location using catheter 214, which can be inserted into a body cavity to reach target tissue 232. In examples, target tissue 232 can comprise a stomach such that target tissue 232 comprises the interior of the stomach and the body cavity can comprise a duct of the esophagus. Catheter 214 can apply hemostat material to the esophagus while being delivered to the stomach, as well as to the stomach when inserted into the stomach. Catheter 214 can include a hemostat material deliver channel or passageway extending therein. Catheter 214 can be directly inserted into anatomy to reach target tissue 232 or can be inserted with the assistance of a guide scope, such as endoscope 14 of FIGS. 3 and 4 and endoscope 104 of FIG. 6A. The endoscope can be steered to guide catheter 214 toward target tissue 232. Imaging capabilities of the endoscope can be used to guide catheter 214 toward target tissue 232. Catheter 214 can be extended from the endoscope to reach the location of target tissue 232. The endoscope can be operated to point catheter 214 toward target tissue 232, such as by using pull wires or steering capabilities. As mentioned, catheter 214 can comprise a separate instrument extending from an endoscope. However, catheter 214 or equivalent hemostat material dispensing capabilities thereof can be integrated directly into an endoscope such that hemostat material 230 can be emitted directly from the endoscope. Additionally, hemostat instrument 108 can be provided alongside endoscope 104. Catheter 214 can be selectively operated by a surgeon or user to spray, dispense, emit or otherwise release hemostat material 230 in the form of gas, liquid, gel, powder, plasma, light or other forms. Catheter 214 can be configured to spray hemostat material 230 in a triangular or conical pattern. In examples, the spray diameter of hemostat material 230 can be adjustable to adjust the spray pattern to the size of the target tissue, as discussed with reference to FIG. 6C. Furthermore, as discussed herein, hemostat material 230 can be drawn in the direction of target tissue 232 via the attraction of positively charged hemostat material 230 toward negatively charged target tissue 232. As such, hemostat material 230 can be drawn out of the air within the endoluminal space to prevent or inhibit “white-out” effect and reduce or eliminate build-up of hemostat material 230 on catheter 214. The present disclosure additionally describes other capabilities for aiming or directionally orienting surgical material onto a desired target anatomy, such as the use of charged gels, strategically shaped and positioned anatomic electrodes, extendable and steerable anatomic electrodes, electrode caps and others. These aiming techniques and devices can reduce surgical material waste and can reduce the time it takes to apply surgical material to an intended target anatomy.

FIG. 3 is a schematic diagram of endoscopy system 10 comprising imaging and control system 12 and endoscope 14. System 10 of FIG. 3 is an illustrative example of an endoscopy system suitable for use with the systems, devices and methods described herein for delivering hemostat material to manage internal bleeding at a surgical site. System 10 can be used to perform various procedures, such as colonoscopy procedures, bariatric producers, and the like, that can be used for removing and obtaining tissue or other biological matter from a patient for analysis or treatment of the patient. According to some examples, endoscope 14 can comprise endoscope 104 of FIGS. 6A-6C and can be insertable into an anatomical region for imaging and/or to provide passage of one or more collection devices for biopsies, or one or more therapeutic devices for treatment of a disease state associated with the anatomical region. Endoscope 14 can, in advantageous aspects, interface with and connect to imaging and control system 12. In the illustrated example, endoscope 14 comprises an end-viewing endoscope, though other types of endoscopes can be used with the features and teachings of the present disclosure.

Imaging and control system 12 can comprise control unit 16, output unit 18, input unit 20, light source unit 22, fluid source 24 and suction pump 26. Imaging and control system 12 can additionally include hemostat material delivery system 200 and electrostatic guide system 202 of FIG. 1.

Imaging and control system 12 can include various ports for coupling with endoscopy system 10. For example, control unit 16 can include a data input/output port for receiving data from and communicating data to endoscope 14. Light source unit 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like. Additionally, control unit 16 can be couplable to leads 218A and 218B of FIG. 1 to electrify leads 218A and 218B. In examples, control unit 16 can produce positive and negative charges on leads 218A and 218B, respectively. Thus, control unit 16 can include receptacles for receiving proximal ends of leads 218A and 218B, such as via plugs or the like. As discussed herein, the charges produced by control unit 16 can apply a first polarity of charge to hemostat material and a second polarity of charge to tissue using various electrodes, such as an external pad, internal gel, an electrode delivered through a scope, an electrode attached to the exterior of a scope, and an electrode comprising a trocar device.

Endoscope 14 can comprise insertion section 28, functional section 30 and handle section 32, which can be coupled to cable section 34 and coupler section 36. Coupler section 36 can be connected to control unit 16 to connect to endoscope 14 to multiple features of control unit 16, such as input unit 20, light source unit 22, fluid source 24 and suction pump 26. Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by pull wires connected to control knob 38 on handle section 32) to maneuver the distal end through tortuous anatomical passageways (e.g., stomach, duodenum, kidney, ureter, colon, etc.). Knob 38 and such pull wires can additionally be used to aim hemostat material using the hemostat delivery systems and hemostat guide systems described herein, such as by bending the shaft of hemostat delivery devices to aim the trajectory of the hemostat material. Insertion section 28 can also include one or more working channels (e.g., an internal lumen) that can be elongate and support insertion of one or more therapeutic tools of functional section 30, such medical instrument 106 of FIG. 6A, a tissue separator device such as forceps, catheter 214 of FIG. 1, hemostat instrument 108 of FIG. 6A, or another medical instrument. The working channels can extend between handle section 32 and functional section 30. Additional functionalities, such as fluid passages, guide wires, and pull wires can also be provided by insertion section 28 (e.g., via suction or irrigation passageways, and the like).

Handle section 32 can comprise knob 38 as well as port 40A. Knob 38 can be coupled to a pull wire, or other actuation mechanisms, extending through insertion section 28. Port 40A, as well as other ports, such as port 40B (FIG. 4), can be configured to couple various electrical cables, guide wires, auxiliary scopes, tissue collection devices, fluid tubes and the like to handle section 32 for coupling with insertion section 28. For example, medical instrument 106 can be fed into endoscope 14 via port 40A. Likewise, catheter 214 of FIG. 1 or hemostat instrument 108 of FIG. 6A can be fed into port 40A or another similar port. Handle section 32 can further comprise control features, such as buttons or levers, for electrically activating leads 218A and 218B (FIG. 1) and activating motive device 124 (FIG. 6A).

Imaging and control system 12, according to examples, can be provided on a mobile platform (e.g., cart 41) with shelves for housing light source unit 22, suction pump 26, image processing unit 42 (FIG. 4), etc. Alternatively, several components of imaging and control system 12 shown in FIGS. 3 and 4 can be provided directly on endoscope 14 so as to make the endoscope “self-contained.”

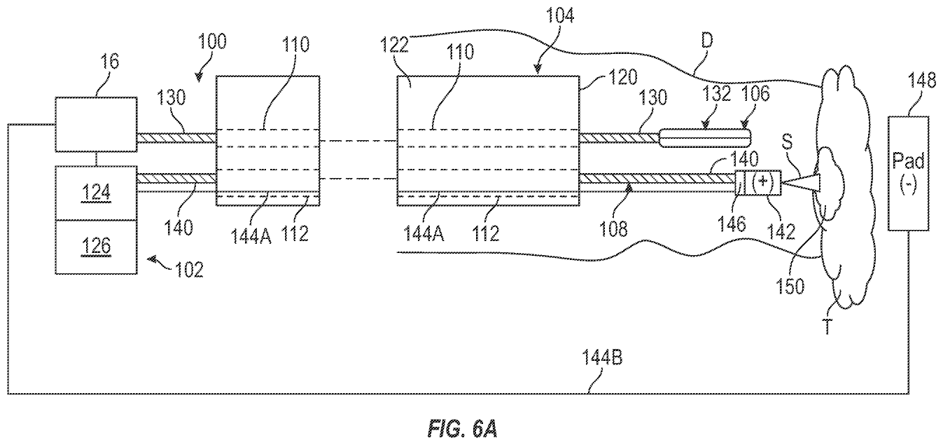

Functional section 30 can comprise components for treating and diagnosing anatomy of a patient. Functional section 30 can comprise an imaging device, an illumination device and an elevator. Functional section 30 can comprise imaging and illuminating components configured for end-viewing, e.g., viewing distally or axially beyond of functional section 30, such as is described further with reference to camera module 70 of FIGS. 5A and 5B.

FIG. 4 is a schematic diagram of endoscopy system 10 of FIG. 3 comprising imaging and control system 12 and endoscope 14. FIG. 4 schematically illustrates components of imaging and control system 12 coupled to endoscope 14, which in the illustrated example comprises an end-viewing colonoscope. Imaging and control system 12 can comprise control unit 16, which can include or be coupled to image processing unit 42, treatment generator 44 and drive unit 46, as well as light source unit 22, input unit 20 and output unit 18. Coupler section 36 can be connected to control unit 16 to connect to endoscope 14 to multiple features of control unit 16, such as image processing unit 42 and treatment generator 44. In examples, port 40A can be used to insert another instrument or device, such as a daughter scope or auxiliary scope, into endoscope 14. Such instruments and devices can be independently connected to control unit 16 via cable 47 (e.g., cable 47 can comprise catheter 214 of FIG. 1 or hemostat instrument 108 of FIG. 6A or a portion thereof). In examples, port 40B can be used to connect coupler section 36 to various inputs and outputs, such as video, air, light and electric. As is discussed below in greater detail with reference to FIGS. 6A-6C, control unit 16 can comprise, or can be in communication with, electrostatic guide system 202. Control unit 16 can be configured to activate a camera to view target tissue distal of endoscope 14. Likewise, control unit 16 can be configured to activate light source unit 22 to shine light on surgical instruments extending from endoscope 14.

Image processing unit 42 and light source unit 22 can each interface with endoscope 14 (e.g., at functional section 30) by wired or wireless electrical connections. Imaging and control system 12 can accordingly illuminate an anatomical region, collect signals representing the anatomical region, process signals representing the anatomical region, and display images representing the anatomical region on output unit 18, which can comprise a cathode ray tube, an LCD display, an LED display and other graphical user interfaces. Imaging and control system 12 can include light source unit 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 3) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

Treatment generator 44 can be configured to generate energy for the performance of medical procedures and hemostat material guide systems. For example, treatment generator 44 can generate acoustic or electrical generator for ablating or cauterizing tissue. Treatment generator 44 can be configured to generate alternating current or direct current power for charging leads 218A and 218B (FIG. 1). Treatment generator 44 can comprise an electrosurgical unit (ESU) monopolar generator, such as an ESG-400 commercially available from Olympus Surgical Technologies. As such, treatment generator 44 can comprise generator 216 (FIG. 1).

FIGS. 5A and 5B illustrate an example of functional section 30 of endoscope 14 of FIG. 4. FIG. 5A illustrates an end view of functional section 30 and FIG. 5B illustrates a cross-sectional view of functional section 30 taken along section plane 5B-5B of FIG. 5A. FIGS. 5A and 5B each illustrate “end-viewing endoscope” (e.g., gastroscope, colonoscope, cholangioscope, etc.) camera module 70. In end-viewing endoscope camera module 70, illumination and imaging systems are positioned such that the viewing angle of the imaging system corresponds to a target anatomy located adjacent (e.g., distal of) an end of endoscope 14 and in line with central longitudinal axis A1 of endoscope 14.

In the example of FIGS. 5A and 5B, end-viewing endoscope camera module 70 can comprise housing 72, working channel 74, fluid outlets 76, illumination lens 78 and objective lens 80. Housing 72 can comprise and endcap for insertion section 28, thereby providing a seal to lumen 82.

As can be seen in FIG. 5B, insertion section 28 can comprise lumen 82 through which various components can be extended to connect functional section 30 with handle section 32 (FIG. 4). For example, illumination lens 78 can be connected to light transmitter 84, which can comprise a fiber optic cable or cable bundle extending to light source unit 22 (FIG. 4). Likewise, objective lens 80 can be coupled to imaging unit 87, which can be coupled to wiring 88. Also, fluid outlets 76 can be coupled to fluid lines 89, which can comprise a tube extending to fluid source 24 (FIG. 3). In examples, one of fluid outlets 76 can comprise an inlet connected to a fluid line 89 configured for suction, such as being connected to a vacuum, for recovery of lavage and irrigation fluid. Other elongate elements, e.g., tubes, wires, cables, can extend through lumen 82 to connect functional section 30 with components of endoscopy system 10, such as suction pump 26 (FIG. 3) and treatment generator 44 (FIG. 4). For example, working channel 74 can comprise a wide-diameter lumen for receiving other treatment components, such as cutting devices and therapeutic devices including medical instrument 106.

Endoscope camera module 70 can also include a photosensitive element, such as a charge-coupled device (“CCD” sensor) or a complementary metal-oxide semiconductor (“CMOS”) sensor. In either example, imaging unit 87 can be coupled (e.g., via wired or wireless connections) to image processing unit 42 (FIG. 4) to transmit signals from the photosensitive element representing images (e.g., video signals) to image processing unit 42, in turn to be displayed on a display such as output unit 18. In various examples, imaging and control system 12 and imaging unit 87 can be configured to provide outputs at desired resolution (e.g., at least 480p, at least 720p, at least 1080p, at least 4K UHD, etc.) suitable for endoscopy procedures.

As described herein, working channel 74 can be used to deliver medical instrument 106 to target tissue. Working channel 74 can additionally be used to deliver retractable anatomy electrode 302 of FIG. 7. Distal electrode device 400 of FIGS. 8A-9 can fit around 72. Additionally, one of fluid outlets 76 can be configured to provide hemostat material, either directly or via insertion of catheter 214 (FIG. 1) therethrough.

FIG. 6A is a schematic view of endoscopy system 100 connected to hemostat material delivery system 102. Endoscopy system 100 can comprise endoscope 104, medical instrument 106 and hemostat instrument 108. FIG. 6B is a perspective view of endoscope 104 showing working channel 110 with medical instrument 106, hemostat channel 112, objective lens 114, illumination lens 116, and fluid outlet 118 in distal end face 120 of elongate shaft 122. FIG. 6C is a perspective view of hemostat instrument 108 showing dispenser 142 having orifice 143. Hemostat material delivery system 102 can comprise motive device 124 and hemostat material reservoir 126. In examples, hemostat material delivery system 102 can comprise conductor 144A, conductor 144B, electrode 146 and pad 148. Conductor 144A, conductor 144B, electrode 146 and pad 148 can be configured as an electrostatic hemostat guide system. FIGS. 6A-6C are discussed concurrently.

In examples, hemostat instrument 108 can comprise catheter 214 of FIG. 1 and endoscope 104 can comprise endoscope 14 of FIGS. 3 and 4. Endoscope 104 can be inserted into anatomic duct D to reach target tissue T.

Medical instrument 106 can be inserted into working channel 110 to obtain tissue from the patient. Thus, medical instrument 106 can cause bleeding of target tissue T. Hemostat instrument 108 can be inserted into hemostat channel 112 to deliver a hemostat material to target tissue T. Hemostat instrument 108 can be used to stop bleeding in target tissue T caused by medical instrument 106 or from other causes. Pad 148 can be positioned proximate target tissue T and can be located outside of anatomic duct D. Pad 148 can comprise pad 220A or pad 220B of FIG. 1. Electrode material 150 can be applied to target tissue T within duct D. Hemostat instrument 108 and medical instrument 106 can be inserted into and withdrawn from endoscope 104 before or after insertion of endoscope 104 from duct D.

Objective lens 114 can be configured similarly as objective lens 80 of FIGS. 5A and 5B. Objective lens 114 can be configured to direct light toward an imaging unit to provide digital images to output unit 18. Illumination lens 116 can be configured similarly as illumination lens 78 of FIGS. 5A and 5B. Illumination lens 116 can be configured to direct light from a light transmitter, such as a light transmitter that receives light from light source unit 22, toward tissue distal of distal end face 120, thereby illuminating tissue for medical instrument 106 and hemostat instrument 108. Fluid outlet 118 can be configured similarly as fluid outlets 76 of FIGS. 5A and 5B. One or more fluid outlets 118 can be configured to deliver and recover fluids, such as by being coupled to a fluid source or a suction source. Specifically, fluid outlet 118 can be connected to fluid source 24 and suction pump 26 (FIG. 3). In examples, fluid outlet 118 can be used to deliver hemostat material, such as by providing fluid source 24 with hemostat material. In examples, a hemostat packet can be added to fluid source 24 at a desired point in time during a procedure to provide hemostat capabilities. The hemostat packet can be manually added to fluid source 24 or can be added to fluid source 24 by an automatic dispensing mechanism of control unit 16 (FIG. 3).

Elongate shaft 122 of endoscope 104 can additionally be provided with steering capabilities as is described with reference to endoscope 14. For example, elongate shaft 122 can include pull wires that can be coupled to an actuation device to impart curvature to elongate shaft 122, thereby allowing endoscope 104 to aim the trajectory of hemostat material emanating from hemostat instrument 108.

Working channel 110 and hemostat channel 112 can be configured to receive a working tool, such as medical instrument 106, and a hemostat delivery system, such as hemostat instrument 108, respectively. Working channel 110 and hemostat channel 112 can extend from distal end face 120 to a proximal portion of elongate shaft 122. For examples, proximal ends of working channel 110 and hemostat channel 112 can each be coupled to a port, such as ports similar to port 40A of FIG. 4, configured to allow a working tool to enter elongate shaft 122. The cross-sectional area or diameter of working channel 110 and hemostat channel 112 can be sized to allow for medical instrument 106 and hemostat instrument 108 to pass freely therethrough, respectively.

Medical instrument 106 can comprise a tissue retrieval device such as a forceps or any other device suitable for separating, retrieving or collecting sample biological matter. Medical instrument 106 can comprise shaft 130 and tissue separators 132. Shaft 130 of medical instrument 106 can comprise a pliable body that can allow tissue separator 132 to be angled out of elongate shaft 122. Shaft 130 can additionally accommodate passage of control features, such as actuation wires, to tissue separator 132 to facilitate actuation of tissue separator 132 to collect tissue.

Hemostat instrument 108 can be configured to deliver a hemostat material, e.g., a clotting agent, such as a gas, liquid or powder, stored in hemostat material reservoir 126. Hemostat instrument 108 can comprise elongate body 140, such as a tube or hose having a lumen through which hemostat material can flow or be dispensed. In examples, elongate body 140 can be open at a distal end to allow hemostat material to flow freely therefrom. In example, hemostat instrument 108 can comprise dispenser 142. Dispenser 142 can comprise a device for controlling or shaping flow of hemostat material from elongate body 140, such as a nozzle and the like. Examples of dispenser 142 are discussed in greater detail with reference to FIG. 6C. In additional examples, elongate body 140 can comprise gas tubes extending between hemostat material delivery system 102 and dispenser 142, and dispenser 142 can comprise an electrode for electrostatic dispensing of hemostat material from dispenser 142. A proximal end portion of hemostat instrument 108 can be connected to one or all of hemostat material delivery system 102, fluid source 24 and control unit 16 (FIG. 3). Motive device 124 can be operated via a user input to obtain hemostat substance from reservoir 126 and provide hemostat substance to dispenser 142. In examples, motive device 124 can comprise a fluid pump, a compressor and the like. Elongate body 140 can be steered or curved via operation of elongate shaft 122 of endoscope 104 to direct hemostat substance S onto target tissue T. Thus, a user of endoscopy system 100 can operate hemostat material delivery system 102 and endoscope 104 to selectively dispense a hemostat material, substance, media or agent to internal anatomic areas during a procedure.

In examples, hemostat instrument 108 can be omitted and hemostat substance can be provided by hemostat material delivery system 102 directly to hemostat channel 112. In such examples, hemostat channel 112 can comprise a leak-proof passage such as a tube or conduit that can convey liquid or powder hemostat material from hemostat material reservoir 126 to dispenser 142.

In examples, hemostat instrument 108 can be provided on the exterior of shaft 122 and hemostat channel 112 can be omitted.

In various examples of delivering hemostat material to the distal end portion of elongate body 140, an electrostatic guide system can be used to charge the hemostat material before or after exiting elongate body 140. Conductor 144A can extend along elongate body 140. In examples, conductor 144A can extend within a passage internal to elongate body 140. In examples, conductor 144A can extend along the exterior of elongate passage and can be attached thereto by various means such as straps, bands or a sheath.

Conductor 144A can be used to deliver electrification to one or more location of elongate body 140 or along a length of elongate body 140. Conductor 144A can be connected to one or both of electrode 146 and dispenser 142 to charge hemostat material travelling through elongate body 140. Thus, as the hemostat material can have a charge when leaving orifice 143 of dispenser 142 (FIG. 6C). In such examples, hemostat material can be charged via corona charging effects.

In examples, hemostat instrument 108 can be configured to generate an electric polarity within anatomy similarly as devices described in Pat. No. U.S. Pat. No. 11,020,166 to Batchelor et al., titled “Multifunctional Medical Device,” which is assigned to Gyrus ACMI, Inc., the contents of which are incorporated herein by this reference.

In additional examples, hemostat material can be charged via triboelectric charging effects. In such examples, hemostat material can be charged via friction of the hemostat material with the hemostat material passageway within elongate body 140. In examples, the hemostat material passageway can be made from or lined with Polytetrafluoroethylene (PTFE) to facilitate the generation of charge. Elongate body 140 can be grounded to prevent charge accumulating on hemostat instrument 108. Hemostat instrument 108 can be grounded via coupling to control unit 16. Triboelectrically charged particles can be used in conjunction with grounded target anatomy or oppositely charged target anatomy.

Pad 148 can function similarly as pads 220A and 220B of FIG. 1. Electrode material 150 can comprise material that can have a native electric charge or that can be used to focus or enhance the charge provided by pad 148. In examples, electrode material 150 can comprise a polymeric gel having conductive metallic particles suspended therein. The gel can adhere to anatomy to enhance the electric field produced by pad 148. Also, the conductive particles within gel comprising electrode material can be pre-charged before delivery to target tissue T. Thus, electrode material 150 can be used to focus the flow of charged hemostat material from hemostat instrument 108 to a specific location, thereby preventing the hemostat material from dispersing into duct D. Electrode material 150 can be delivered to target tissue T using a delivery catheter, such as commercially available single-use spray catheters that can be inserted into endoscope 104. Additionally, electrode material 150 can be placed manually in open procedures. Electrode material 150 can be place in places where bleeding or hemorrhaging is occurring or where an incision or tissue-removal procedure is to occur in anticipation of hemorrhaging or bleeding.

With particular reference to FIG. 6C, dispenser 142 can comprise a nozzle or another device for controlling the pattern of spray S by adjusting the size of orifice 143. For example, dispenser 142 can be adjusted by an operator, such as at control unit 16 (FIGS. 1 and 2), to adjust the spray density, spray pattern, spray diameter, spray distance and the like. For example, dispenser 142 can be adjusted to produce spray pattern S1 for dispensing hemostat material in a concentrated manner over a longer distance or to produce spray pattern S2 for dispensing hemostat material in a dispersed manner of a shorter distance. Dispenser 142 can comprise various types of devices, such as a flat fan nozzle, a hollow cone nozzle, a full cone nozzle, a misting nozzle, a misting nozzle and an air atomizing nozzle. Dispenser 142 can be connected to an actuator or motor that can be used to change the size of orifice 143. Dispenser 142 can be adjusted by a user interface, such as a button or lever, located at handle section 32.

FIG. 7 is a perspective view of hemostat instrument 300 suitable for use with electrostatic hemostat material delivery system 200 of the present disclosure comprising retractable anatomy electrode 302. Hemostat instrument 300 can comprise an endoscope including working channel 310, hemostat channel 312, objective lens 314, illumination lens 316, and electrode outlet 318 in distal end face 320 of shaft 322. Hemostat instrument 300 can be configured similarly as endoscope 104 of FIG. 6A, except retractable anatomy electrode 302 can be additionally used therein. Thus, hemostat spray 324 can be generated using any of the devices and methods described herein to generate charged hemostat powder. Target tissue can be charged with an opposite polarity as hemostat spray 324 using retractable anatomy electrode 302. Retractable anatomy electrode 302 can comprise conductor 330, insulation jacket 332 and expandable pad 334, which can comprise first portion 336A and second portion 336B. Electrode 302 can be coupled to an electrical generator, such as generator 216 of FIG. 1. Insulation jacket 332 can extend along the majority of retractable anatomy electrode 302 to prevent contact with other portions of hemostat instrument 300. A distal portion of retractable anatomy electrode 302 can be uninsulated for coupling to expandable pad 334. Expandable pad 334 can be used in increase the contact surface area of electrode 302 with target tissue T. Expandable pad 334 can be configured to expand radially outward from conductor 330. In examples, expandable pad 334 can comprise a metallic mesh cone such that portions 336A and 336B can expand radially to form a circular on elliptical surface area to contact target tissue T. In examples, expandable pad 334 can comprise a metallic toggle joint such that portions 336A and 336B can pivot outward to form a rectangular or oblong surface are to contact target tissue T. Thus, expandable pad 334 can moved distally from electrode outlet 318 to radially expand, but can be drawn proximally into electrode outlet 318 to radially collapse. Expandable pad 334 can be self-expanding. Portions 336A and 336B can comprise a porous structure or a mesh structure having openings that allow hemostat powder to pass therethrough, but that provide contact with target tissue T at a plurality of locations to provide an electric field with a large surface area to cover bleeding or hemorrhaging tissue. For example, expandable pad 334 can comprise a grid of wires spaced at wide intervals, wherein the grid of wires can comprise electrification over a large surface area of tissue and the spaces therebetween can allow hemostat material to reach the tissue. Additionally, expandable pad 334 can comprise a blunt tip for conductor 330 to prevent unintended puncture of target tissue T. In examples, expandable pad 334 can be replaced by or include a blunted tip, such as a bulb.

FIG. 8A is a schematic exploded view of a distal electrode device 400 configured for placement at a distal end of scope 402. FIG. 8B is a schematic assembled view of distal electrode device 400 of FIG. 8A attached to scope 402. FIGS. 8A and 8B are discussed concurrently.

Distal electrode device 400 can comprise body 404, conductor 406, side port 408, rim 410 and opening 412. Scope 402 can comprise working channel 414, objective lens 416, illumination lens 418, shaft 420 and end face 422. Scope 402 can be configured similarly as endoscope 104 of FIG. 6B with only certain elements shown for simplicity.

Body 404 can be configured as an annular body sized to fit over shaft 420. Thus, opening 412 can be approximately the same size or slightly larger than the size of shaft 420. In examples, opening 412 and shaft 420 comprise circular cross-sectional areas. Body 404 can be configured to be positioned along shaft 420, such as by sliding. Rim 410 can be positioned within opening 412 to limit the amount that distal electrode device 400 can be slid proximally away from end face 422. As such end face 424 of body 404 can be positioned a fixed distance away from end face 422. Thus, when end face 424 is pushed against tissue, as shown in FIG. 9, a chamber can be formed between the tissue and end face 422 to capture hemostat material. Rim 410 can comprise an annular ledge extending around body 404 within opening 412. Side port 408 can be located on body 404 distally of rim 410. Side port 408 can allow for fluid communication between the distal end face 422 and the side of body 404 when distal electrode device 400 is pushed against tissue, as described with reference to FIG. 9. In the illustrated example, a single side port 408 is shown, but additional side ports 408 can be included.

Conductor 406 can be attached to body 404 in any suitable fashion such that electricity can be conducted from conductor 406 to body 404. Body 404 can be fabricated of a conducting material, such as metal or stainless steel. In additional examples, body 404 can be fabricated of plastic impregnated with metallic flakes or fibers. Conductor 406 can be electrified, such as by being connected to generator 216 (FIG. 2). In the illustrated example, conductor 406 is configured to extend along the outside of shaft 420. In additional examples, conductor 406 can be positioned inside shaft 420, such as through working channel 414 or another channel or passageway.

FIG. 9 is a schematic view of the distal electrode device 400 attached to distal end face 422 of scope 402 to dispense hemostat material 430 to target tissue 432. Scope 402 can be guided to target tissue 432 via any suitable methods, such as those discussed herein. Scope 402 can be advanced so that end face 424 of body 404 contacts target tissue 432. An electrical charge can be generated in hemostat material 430 via any of the methods described herein. For example, hemostat instrument 108 (FIG. 6A) can be used in working channel 414 to emit hemostat material 430. An electrical charge can be generated at body 404 via electrification of conductor 406. As such, hemostat material 430 and body 404 can be oppositely charged so that hemostat material 430 will be attracted to target tissue 432. Additionally, hemostat material 430 will be attracted to body 404 and thereby also be propelled toward side port 408. Thus, hemostat material 430 can be directionally aimed via rotating scope 402. In additional examples, body 404 can be configured to be independently rotatable relative to scope 402.

FIG. 10 is schematic illustration of electrostatic hemostat material delivery system 440 comprising trocar devices 441 and 442 that can be configured to generate an electrical charge for directionally applying hemostat material. Trocar device 441 can be used with scope 444, which can be connected to image processing unit 42. Trocar device 442 can be used with hemostat instrument 446, which can be connected to hemostat material reservoir 126. Hemostat material reservoir 126 and image processing unit 42 can be part of control unit 16 (FIG. 4). As such, any of the instruments shown in FIG. 10 can additionally be connected to treatment generator 44 (FIG. 4) to produce an electrical charge at such instrument. In the illustrated example, trocar device 441 can be configured to produce a negative charge on patient P and hemostat instrument 446 can be used to produce positively charged hemostat material 448.

Trocar device 441 can be placed through incision 450 in patient P to reach body cavity 452. Trocar device 441 can comprise tubular body 454 having an internal passage that allows scope 444 to be passed into and through trocar device 441 while holding incision 450 in an open position.

Trocar device 442 can be placed through incision 456 in patient P to reach body cavity 452. Trocar device 442 can comprise tubular body 458 and tubular body 460. Tubular body 460 can be inserted into a passage extending through tubular body 458. Tubular body 458 can include an internal passage to receive instrument 462. Tubular body 460 can include an internal passage to receive hemostat instrument 446. Instrument 462 can comprise a clamp forceps or hemostat.

Tubular body 454 and tubular body 458 can include rims or flanges to prevent passage into incisions 450 and 456, respectively. Tubular body 454 and tubular body 458 can further comprise internal sealing means to allow passage of instruments into tubular body 454 and tubular body 458 but inhibit or prevent the passage of biological fluid out of tubular body 454 and tubular body 458.

Either or both of trocar devices 441 and 442 can be connected to treatment generator 44 to provide electrification to adjacent anatomy. Thus, stronger electrical field strength can be produced in tissue adjacent trocar devices 441 and 442. Since trocar devices 441 and 442 can be placed in proximity to target tissue, such as body organ BO, where it can be desirable to use hemostat material, trocar devices 441 and 442 can be used, together or individually, to directionally orient or aim dispensed hemostat material.

FIG. 11 is a perspective view of electrode mat 480 shaped to mate with particular anatomic areas. Electrode mat 480 can comprise spinal panel 482, tailbone panel 484 and lumbar panel 486. Lumbar panel 486 can comprise left side 488A and right side 488B. Electrode mat 480 can thus be shaped to extend along specific portions of a patient, such as the back, e.g., spinal area, of a patient. Spinal panel 482 can be curved to extend along a typical spinal column. Lumbar panel 486 can be configured to extend across the lumbar region of a patient outward of the spinal column to reach areas within an abdomen where organs are located.

Electrode mat 480 can include various couples for connecting to leads 218A and 218B of FIG. 1. In examples, electrode mat 480 can be used as one or both of pads 220A and 220B of FIG. 1. Electrode mat 480 can be made of a conducting material, such as metal. Additionally, electrode mat 480 can be made of an insulating material and can be coated or impregnated with a conducting material. In various examples, the conduction material can be included in electrically isolated circuits contacting different zones of electrode mat 480. Thus, for example, one or both of spinal panel 482, tailbone panel 484 and lumbar panel 486 can be electrified. Similarly, one or both of left side 488A and right side 488B can be included in separately electrifiable zones. As such, only the zones or regions of electrode mat 480 that are in close proximity to target tissue of a patient can be electrified to produce the desired electrical field in the anatomy. For example, a surgeon or technician can adjust a setting or various switches on electrode mat 480 to provide the desired configuration.

FIG. 12 is a cross-sectional view of electrode mat 480 having lumbar panel 486 shaped to conform to anatomic contours. Lumbar panel 486 can comprise spinal portion 490, left pad 492A and right pad 492B. Lumbar panel 486 can be configured to provide three-dimensional depth to electrode mat 480 in particular anatomic regions. In the illustrated example, lumbar panel 486 can be curved to wrap partially around an abdomen of a patient. The three-dimensionally curved portions of lumbar panel 486 including left pad 492A and right pad 492B can comprise individually electrifiable zones that can provide electric polarization proximate a desired anatomic area, such as a kidney or liver.

FIGS. 11 and 12 illustrate particular examples of an electrode mat that can be two-dimensionally and three-dimensionally shaped to mate with specific external surfaces of a patient to be in close proximity to specific internal organs. Other shapes can be used, such as pads shaped to the contour of a buttocks, rib cage or chest area, groin area, neck area and the like. Thus, electrode mat 480 and other example electrode mats can be used to directionally orient or pull hemostat material into a desired direction toward anatomy where the hemostat material is desired and away from the air, surgical instruments and other anatomy.

The devices, systems and methods of the present disclosure can utilize various substances and compositions as the hemostat substance. A variety of formulations are possible and could be in the form of a liquid, gel, or powder. Attributes of the hemostat substance can comprise:
1. Biocompatible with little or no side effects.
2. Hemostat should stay in the area sprayed and provide some visual difference between treated and untreated areas;
3. Visual difference dissipates, does not affect visualization during the procedure, or enhances the visualization during the remainder of the procedure; and
4. Reasonable cost in mass production.

Suitable formulations for the hemostat material can comprise those commonly used as clotting agents, including granules of one or more of a mineral, such as zeolite, and a chitosan. In examples, the hemostat material can comprise a commercially available hemostat agent. In examples, the hemostat material can comprise a polymer, such as adhesive hemostatic polymers. In examples, the hemostat material can comprise a starch, such as a polysaccharide.

In examples, clotting agents suitable for use as hemostat materials for use herein are described in Khoshmohabat, Hadi et al. “Overview of Agents Used for Emergency Hemostasis.” Trauma monthly vol. 21,1 e26023. 6 Feb. 2016, doi:10.5812/traumamon.26023.

In examples, clotting agents suitable for use as hemostat materials for use herein are described in Pub. No. US 2018/0361011 to Norowski Jr., titled “CARBOXYMETHYL CHITOSAN SPONGE FORMULATION,” which is assigned to Gyrus ACMI, Inc., the contents of which are incorporated herein by this reference.

Examples of procedures that can be performed using the systems, devices and methods of the present disclosure include Endoscopic Submucosal Dissection (ESD) procedures. Exemplary ESD procedures are described in Pat. No. U.S. Pat. No. 9,402,683 to Yamano et al., titled “Submucosal layer dissection instrument, submucosal layer dissection system, and submucosal layer dissection method,” which is assigned to Olympus Corporation, the contents of which are incorporated herein by this reference. Although, other types of procedures can be used with the methods, systems and devices of the present disclosure. For example, hemostat material can be applied in emergency bleeding situations in the upper and lower gastrointestinal tract.

### EXAMPLES

Example 1 is a surgical material delivery system comprising: an elongate insertion shaft comprising: a passageway extending at least partially through the elongate insertion shaft; and a discharge opening fluidly connected to the passageway; a first electrode connected to the elongate insertion shaft configured to impart an electrical charge to surgical material flowing through the passageway; and a second electrode connected to the elongate insertion shaft configured to impart an electrical charge to tissue in contact with the second electrode.

In Example 2, the subject matter of Example 1 optionally includes wherein the first electrode comprises a first conductor extending along the elongate insertion shaft for coupling to an electrical generator.

In Example 3, the subject matter of Example 2 optionally includes wherein the first electrode further comprises a ring attached to the elongate insertion shaft.

In Example 4, the subject matter of any one or more of Examples 2-3 optionally include wherein the first electrode further comprises a metallic spray tip connected to the discharge opening.

In Example 5, the subject matter of Example 4 optionally includes wherein the metallic spray tip is configured to produce a variable diameter spray pattern of the surgical material.

In Example 6, the subject matter of any one or more of Examples 1-5 optionally include wherein the second electrode extends from the elongate insertion shaft distally of the discharge opening.

In Example 7, the subject matter of Example 6 optionally includes wherein the second electrode is retractable proximally of the discharge opening.

In Example 8, the subject matter of Example 7 optionally includes wherein the second electrode comprises: a wire having an extension portion and a distal tip; and an insulation jacket extending along the extension portion to leave the distal tip exposed.

In Example 9, the subject matter of Example 8 optionally includes wherein the distal tip includes a collapsible pad.

In Example 10, the subject matter of any one or more of Examples 6-9 optionally include wherein the second electrode comprises a cap couplable to a distal end face of the elongate insertion shaft.

In Example 11, the subject matter of Example 10 optionally includes wherein the cap comprises: a cylindrical body configured to circumscribe the elongate insertion shaft; and a side port extending through the cylindrical body to allow surgical material to pass therethrough.

In Example 12, the subject matter of any one or more of Examples 6-11 optionally include wherein the elongate insertion shaft comprises an endoscope.

In Example 13, the subject matter of any one or more of Examples 1-12 optionally include wherein the second electrode comprises a trocar device.

In Example 14, the subject matter of Example 13 optionally includes wherein the trocar device comprises a tubular body into which the elongate insertion body is inserted.

In Example 15, the subject matter of any one or more of Examples 1-14 optionally include a third electrode comprising a pad connectable to an electrical generator via a third conductor.

In Example 16, the subject matter of Example 15 optionally includes wherein the pad comprises a plurality of conducting zones, each conducting zone independently activatable to produce an electric field.

In Example 17, the subject matter of any one or more of Examples 15-16 optionally include wherein the pad comprise a two-dimensional or three-dimensional shape configured to correspond to an anatomic shape.

In Example 18, the subject matter of any one or more of Examples 1-17 optionally include wherein the passageway of the elongate insertion shaft is lined with PTFE.

In Example 19, the subject matter of any one or more of Examples 1-18 optionally include a conducting gel comprising electrically chargeable particles.

In Example 20, the subject matter of any one or more of Examples 1-19 optionally include an electrical generator coupled to the first electrode and the second electrode to impart opposite charges to the first electrode and the second electrode; a surgical material reservoir fluidly connected to the passageway; and a propulsion system fluidly connected to the surgical material reservoir.

Example 21 is a method for delivering a surgical material to an internal lumen of a patient, the method comprising: inserting a delivery device into an anatomic area; coupling a surgical material delivery system to the delivery device, the surgical material delivery system having a reservoir of a surgical material; positioning an anatomic electrode relative to the anatomic area; applying a first charge to the anatomic electrode; dispensing surgical material from the delivery device; applying a second charge to surgical material leaving the delivery device, the second charge opposite the first charge; and delivering charged surgical material to the anatomic area proximate the anatomic electrode via a directional-oriented electrostatic field.

In Example 22, the subject matter of Example 21 optionally includes wherein applying the second charge to surgical material leaving the delivery device comprises electrifying a conductor extending along the delivery device.

In Example 23, the subject matter of Example 22 optionally includes wherein applying the second charge to surgical material leaving the delivery device further comprises electrifying a ring electrode disposed on the delivery device.

In Example 24, the subject matter of any one or more of Examples 22-23 optionally include wherein applying the second charge to surgical material leaving the delivery device further comprises electrifying a spray nozzle disposed on the delivery device.

In Example 25, the subject matter of Example 24 optionally includes modulating a spray diameter of surgical material exiting the delivery device with the spray nozzle.

In Example 26, the subject matter of any one or more of Examples 21-25 optionally include inserting the anatomic electrode into an electrode channel in the delivery device configured to receive the anatomic electrode.

In Example 27, the subject matter of Example 26 optionally includes extending the anatomic electrode distally of the delivery device; and retracting the anatomic electrode proximally of the delivery device.

In Example 28, the subject matter of Example 27 optionally includes wherein extending the anatomic electrode from an endoscope in which the delivery device is disposed comprises: sliding a wire having an extension portion and a distal tip toward the anatomic area; and insulating the wire from the internal lumen with a jacket.

In Example 29, the subject matter of Example 28 optionally includes deploying an expandable pad attached to the distal tip of the wire.

In Example 30, the subject matter of any one or more of Examples 21-29 optionally include attaching the anatomic electrode to a distal end face of the delivery device.

In Example 31, the subject matter of Example 30 optionally includes wherein the anatomic electrode comprises a cap comprising: a cylindrical body configured to circumscribe the delivery device; and a side port extending through the cylindrical body.

In Example 32, the subject matter of any one or more of Examples 21-31 optionally include inserting the delivery device into an endoscope.

In Example 33, the subject matter of any one or more of Examples 21-32 optionally include inserting the anatomic electrode into an incision in anatomy.

In Example 34, the subject matter of Example 33 optionally includes wherein the anatomic electrode comprises a trocar device comprising a tubular body.

In Example 35, the subject matter of any one or more of Examples 21-34 optionally include wherein applying the first charge to the anatomic electrode comprises electrifying a pad disposed outside of the internal lumen.

In Example 36, the subject matter of Example 35 optionally includes wherein positioning the anatomic electrode relative to the anatomic area comprises extending a contour of the anatomic electrode along an anatomic contour.

In Example 37, the subject matter of any one or more of Examples 35-36 optionally include wherein electrifying the pad disposed outside of the internal lumen comprises electrifying less than all of a plurality of conducting zones distributed within the pad.

In Example 38, the subject matter of any one or more of Examples 25-37 optionally include wherein applying the first charge to the anatomic electrode comprises applying a conducting gel to a position inside the internal lumen.

In Example 39, the subject matter of any one or more of Examples 21-38 optionally include electrostatically charging the surgical material within the delivery device using friction.

In Example 40, the subject matter of any one or more of Examples 21-39 optionally include activating a propulsion system connected to the delivery device; and pushing surgical material from a surgical material reservoir fluidly connected to the delivery device.

Each of these non-limiting examples can stand on its own, or can be combined in various permutations or combinations with one or more of the other examples.

### Various Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A surgical material delivery system comprising:
an elongate insertion shaft comprising:
a passageway extending at least partially through the elongate insertion shaft; and
a discharge opening fluidly connected to the passageway;
a first electrode connected to the elongate insertion shaft configured to impart an electrical charge to surgical material flowing through the passageway; and
a second electrode connected to the elongate insertion shaft configured to impart an electrical charge to tissue in contact with the second electrode.

2. The surgical material delivery system of claim 1, wherein the first electrode comprises:
a first conductor extending along the elongate insertion shaft for coupling to an electrical generator; and
a ring attached to the elongate insertion shaft.

3. The surgical material delivery system of claim 1, wherein the first electrode comprises:
a first conductor extending along the elongate insertion shaft for coupling to an electrical generator; and
a metallic spray tip connected to the discharge opening;
wherein the metallic spray tip is configured to produce a variable diameter spray pattern of the surgical material.

4. The surgical material delivery system of claim 1, wherein the second electrode extends from the elongate insertion shaft distally of the discharge opening and is retractable proximally of the discharge opening.

5. The surgical material delivery system of claim 4, wherein the second electrode comprises:
a wire having an extension portion and a distal tip; and
an insulation jacket extending along the extension portion to leave the distal tip exposed;
wherein the distal tip includes a collapsible pad.

6. The surgical material delivery system of claim 1, wherein the second electrode extends from the elongate insertion shaft distally of the discharge opening and comprises a cap couplable to a distal end face of the elongate insertion shaft; wherein the cap comprises:
a cylindrical body configured to circumscribe the elongate insertion shaft; and
a side port extending through the cylindrical body to allow surgical material to pass therethrough.

7. The surgical material delivery system of claim 4, wherein the elongate insertion shaft comprises an endoscope.

8. The surgical material delivery system of claim 1, wherein the second electrode comprises a trocar device, wherein the trocar device comprises a tubular body into which the elongate insertion shaft is inserted.

9. The surgical material delivery system of claim 1, further comprising a third electrode comprising a pad connectable to an electrical generator via a third conductor, wherein the pad comprises a plurality of conducting zones, each conducting zone independently activatable to produce an electric field.

10. The surgical material delivery system of claim 1, further comprising a third electrode comprising a pad connectable to an electrical generator via a third conductor, wherein the pad comprise a two-dimensional or three-dimensional shape configured to correspond to an anatomic shape.

11. The surgical material delivery system of claim 1, wherein the passageway of the elongate insertion shaft is lined with PTFE.

12. The surgical material delivery system of claim 1, further comprising a conducting gel comprising electrically chargeable particles.

13. The surgical material delivery system of claim 1, further comprising:
an electrical generator coupled to the first electrode and the second electrode to impart opposite charges to the first electrode and the second electrode;
a surgical material reservoir fluidly connected to the passageway; and
a propulsion system fluidly connected to the surgical material reservoir.

14. A method for delivering a surgical material to an internal lumen of a patient, the method comprising:
inserting a delivery device into an anatomic area;
coupling a surgical material delivery system to the delivery device, the surgical material delivery system having a reservoir of a surgical material;
positioning an anatomic electrode relative to the anatomic area;
applying a first charge to the anatomic electrode;
dispensing surgical material from the delivery device;
applying a second charge to surgical material leaving the delivery device, the second charge opposite the first charge; and
delivering charged surgical material to the anatomic area proximate the anatomic electrode via a directional-oriented electrostatic field.

15. The method of claim 14, wherein applying the second charge to surgical material leaving the delivery device comprises:
electrifying a conductor extending along the delivery device; and
electrifying a ring electrode disposed on the delivery device.

16. The method of claim 14, wherein applying the second charge to surgical material leaving the delivery device comprises:
electrifying a conductor extending along the delivery device;
electrifying a spray nozzle disposed on the delivery device; and
modulating a spray diameter of surgical material exiting the delivery device with the spray nozzle.

17. The method of claim 14, further comprising:
inserting the anatomic electrode into an electrode channel in the delivery device configured to receive the anatomic electrode;
extending the anatomic electrode distally of the delivery device; and
retracting the anatomic electrode proximally of the delivery device.

18. The method of claim 17, wherein extending the anatomic electrode from an endoscope in which the delivery device is disposed comprises:
sliding a wire having an extension portion and a distal tip toward the anatomic area;
insulating the wire from the internal lumen with a jacket; and
deploying an expandable pad attached to the distal tip of the wire.

19. The method of claim 14, further comprising attaching the anatomic electrode to a distal end face of the delivery device;
wherein the anatomic electrode comprises a cap comprising:
a cylindrical body configured to circumscribe the delivery device; and
a side port extending through the cylindrical body.

20. The method of claim 14, further comprising inserting the delivery device into an endoscope.

21. The method of claim 14, further comprising inserting the anatomic electrode into an incision in anatomy, wherein the anatomic electrode comprises a trocar device comprising a tubular body.

22. The method of claim 14, wherein applying the first charge to the anatomic electrode comprises electrifying a pad disposed outside of the internal lumen and extending a contour of the anatomic electrode along an anatomic contour.

23. The method of claim 14, wherein applying the first charge to the anatomic electrode comprises electrifying a pad disposed outside of the internal lumen and electrifying less than all of a plurality of conducting zones distributed within the pad.

24. The method of claim 14, wherein applying the first charge to the anatomic electrode comprises applying a conducting gel to a position inside the internal lumen.

25. The method of claim 14, further comprising electrostatically charging the surgical material within the delivery device using friction.

26. The method of claim 14, further comprising:
activating a propulsion system connected to the delivery device; and
pushing surgical material from a surgical material reservoir fluidly connected to the delivery device.
